(19) United States
(12) Patent Application Publication
(10) Pub. No.: US 2020/0216790 A1
(43) Pub. Date: Jul. 9, 2020

(54) MICROFLUIDIC DEVICE FOR CELL CULTURE EXPERIMENTS AND USES THEREOF

(71) Applicants: FRAUNHOFER-GESELLSCHAFT ZUR FÖRDERUNG DER ANGEWANDTEN FORSCHUNG E. V., München (DE); TECHNISCHE UNIVERSITÄT DRESDEN, Dresden (DE)

(72) Inventors: Peter LOSKILL, Stuttgart (DE); Christopher PROBST, Stuttgart (DE); Mathias BUSEK, Dresden (DE); Stefan GRÜNZNER, Dresden (DE); Frank SONNTAG, Dresden (DE)

(73) Assignees: FRAUNHOFER-GESELLSCHAFT ZUR FÖRDERUNG DER ANGEWANDTEN FORSCHUNG E. V., München (DE); TECHNISCHE UNIVERSITÄT DRESDEN, Dresden (DE)

(21) Appl. No.: 16/648,279

(22) PCT Filed: Sep. 17, 2018
(86) PCT No.: PCT/EP2018/075090

§ 371 Date: Mar. 18, 2020

(30) Foreign Application Priority Data
Sep. 19, 2017 (DE) 10 2017 216 581.2

(51) Int. Cl. C12M 3/06; C12M 1/00; C12M 1/34; C12M 1/36

(52) U.S. Cl. CPC C12M 23/16; C12M 41/48; C12M 41/46; C12M 29/00

(57) ABSTRACT

The invention relates to a microfluidic device which has at least one first culture chamber with cardiac muscular cells, at least one microfluidic channel, at least one pump (for example a micropump), and at least one detector, said detector being configured to detect an activity of the cardiac muscular cells contained in the culture chamber. The microfluidic device additionally contains at least one controller which is configured to control the at least one pump on the basis of the cardiac muscular cell activity detected by the at least one detector. The invention additionally relates to uses of the microfluidic device.

In accordance with the invention, a microfluidic device is presented, which has at least one first culture chamber with cardiomyocytes, at least one microfluidic channel, at least one pump (e.g. a micropump) and at least one detector, wherein the detector is configured to detect activity of the cardiomyocytes contained in the culture chamber. The microfluidic device further comprises at least one control device, which is configured to control the at least one pump based on an activity of the cardiomyocytes detected by the at least one detector. In addition, uses of the microfluidic device are proposed.

Throughout the world, many teams are engaged in the development of artificial heart tissue. Especially for the testing of substances without animal experiments, new impetus is expected here, because with cell-based systems a patient-oriented, individualized search for active substances is possible. Existing in-vitro platforms are generally of too simple construction (2-dimensional, no perfusion, microenvironment absent) to recreate the physiological behavior of heart tissue adequately. There is therefore a need for microfluidic devices, also called microphysiological systems (abbreviated to: MPSs), which reproduce the microenvironment and offer 3-dimensional tissue spaces, which come closer to human tissue. The aim is on the one hand to construct organ models that are as close as possible to the in-vivo situation, and on the other hand the culture of several such models in a closed circulating system, so as to be able to conduct systemic testing of active substances thereon (so-called “multiorgan chip”).

Under physiological conditions, the cardiomyocytes begin with spontaneous or (electrically) stimulated movement, which may be influenced by the prevailing microenvironment (hypoxia, nutrient deficiency), metabolic products or the addition of substances. Beat frequency, contraction velocity (or relaxation velocity), contractile force, contraction time, rhythmicity and maximum deformation are pharmacologically relevant parameters, which can be determined for example by microscopic observation, for example with subsequent video analysis. Starting from these parameters, the human body possesses a complex control mechanism, which is lacking in existing MPSs. For example, existing microfluidic devices are unable to show that, because the heart supplies itself, it is itself more poorly supplied when its pumping capacity declines (which in its turn may lead to a further decline in pumping capacity).

A number of different heart muscle in-vitro systems are already known in the prior art, which differ in size (individual cells to macroscopic 3D tissues), origin (e.g. primary rodent cells, cell lines, based on stem cells), structure (isotropic or anisotropic) as well as the implementation of perfusion. In most cases replication of the heart muscle is then focused on its use as an “electromechanical sensor”, for directly determining the effects of drugs or stimuli on the tissue. In just a few studies the reproduced heart muscle tissues were actually used for producing perfusion. Owing to the small forces generated by the micro-heart muscles, direct production of perfusion (cardiomyocyte pump) is not practicable owing to the high fluidic resistance of microchannels.

Readout of contractile movements or of the contractile forces is accomplished in various ways in the heart muscle in-vitro systems in the prior art. For example, flexible spring beams, 3D-printed strain gauges and optical analysis methods are used for this. Electrophysiological sensors, such as for example microelectrode arrays (MEAS), Patch-Clamp, “sharp electrodes” and calcium imaging are also used.

A circulation system is known from DE 10 2013 011 768 A1, and a method is presented for the vital supply of cell cultures in a microfluidic network. In this microfluidic network there is defined supply of several cell culture compartments via throttles and/or valves. With these throttles and/or valves it is possible for example to set and control the pH, oxygen partial pressure, carbon dioxide partial pressure and/or glucose concentration.

Multilayer microfluidic devices with integrated active components (such as for example pumps and oxygenators) for direct culture of cardiomyocytes have already been made.

Furthermore, a module with a feedback loop for controlling and setting defined gas compositions is known in the prior art.

Up to now, the MPSs with cardiomyocytes known in the prior art are characterized by the disadvantage that the influence of the activity of cardiomyocytes on the fluid stream in the microfluidic device is represented by them inadequately, if at all. In other words the known systems or devices do not allow a defined activity of the cardiomyocytes present at a defined time point to be translated practically immediately (i.e. without temporal delay) into a fluid stream corresponding to the present activity in the system, as is mediated perfectly well in-vivo by the control mechanism of the cardiovascular system. As a result of this disadvantage, with currently known microfluidic devices with heart cells, the true in-vivo state of a living patient can only be simulated inadequately, because in a living patient a change in the activity of the cardiomyocytes may sometimes have very considerable effects on the fluid stream of the blood circulation and therefore the blood and oxygen supply of individual cells and organs.

Accordingly, the aim of the present invention was to provide a microfluidic device with cardiomyocytes, which permits a more accurate and more realistic investigation of the effect of certain factors on the activity of cardiomyocytes and in addition makes it possible to detect the activity of other biological cells (e.g. liver cells or nerve cells) as realistically as possible as a function of the altered cardiomyocyte activity (e.g. under the conditions of arrhythmia or cardiogenic shock).

This aim is achieved by the microfluidic device with the features of claim 1 and the uses as claimed in claim 23. The dependent claims present advantageous variants.

According to the invention, a microfluidic device is provided, comprising
a) at least one first culture chamber containing cardiomyocytes;
b) at least one microfluidic channel;
c) at least one pump (e.g. a micropump) for pumping a liquid through the at least one microfluidic channel and the at least one culture chamber;
d) at least one detector, which is configured to detect an activity of the cardiomyocytes contained in the culture chamber;
   characterized in that the microfluidic device further comprises at least one control device which is configured to control the at least one pump based on the activity of the cardiomyocytes detected by the at least one detector.

The term “activity of the cardiomyocytes” means in particular electrical activity and/or movement activity of the cardiomyocytes (preferably both), wherein the term also comprises electrical activity of zero (no action potential) and movement activity of zero (absence of movement). The “movement activity of the cardiomyocytes” means in particular the contractility and/or the frequency of movement of the cardiomyocytes (preferably both).

In the device according to the invention, the activity of cardiomyocytes can be detected and, via a feedback loop to the pump, the amount of fluid delivered (perfusion) can be adapted corresponding to the activity of the cardiomyocytes. By this measure, not only the influence of altered perfusion on the cardiomyocytes themselves be studied more realistically, but so too can the influence of altered perfusion on other biological cells, which are located in further culture chambers in the microfluidic device.

Owing to the feedback loop, the complex control mechanism of the cardiovascular system of living organisms can be better reproduced than in existing systems or devices from the prior art. In other words, with the device according to the invention it is possible to make a statement that is more realistic and more relevant to the real in-vivo situation, regarding the effect that certain factors have on the cardiovascular system. Owing to the basic possibility of circulating the liquid in the microfluidic device it becomes possible moreover to predict the risk of time-delayed sequelae more appropriately, as certain substances or metabolic products can have effects over a long period.

Moreover, with the device according to the invention it is possible to reproduce the effect of altered activity of cardiomyocytes on further tissue types (e.g. liver tissue, nerve cells etc.) realistically, which is of decisive importance especially for systemic testing of (e.g. chemical, biochemical or biological) substances.

The microfluidic device may be characterized in that the at least one control device is configured to increase the pumping capacity of the at least one pump when there is strong activity of the cardiomyocytes, preferably by increasing the pumping frequency and/or pump lift. Furthermore, the at least one control device may be configured to decrease the pumping capacity of the at least one pump when there is weak activity of the cardiomyocytes, preferably by decreasing the pumping frequency and/or pump lift.

The at least one control device may further be configured to reduce or to increase perfusion of the at least one first culture chamber containing cardiomyocytes, preferably by opening or closing a by-pass channel connected fluidically parallel to the at least one first culture chamber. Furthermore, the at least one control device may be configured to reduce or to increase perfusion of at least one second culture chamber in the microfluidic device, preferably by opening or closing a by-pass channel connected fluidically parallel to the at least one second culture chamber.

The at least one first culture chamber may contain cardiomyocytes which form at least one heart muscle fiber, wherein the cardiomyocytes of the at least one heart muscle fiber are preferably oriented anisotropically. Moreover, the culture chambers may contain biological cells that differ from cardiomyocytes, preferably cells selected from the group consisting of fibroblasts, endothelial cells and combinations thereof. Furthermore, the cardiomyocytes may comprise, or be embedded in, a hydrogel.

In a preferred embodiment, the microfluidic device has at least one reservoir, which contains a nutrient solution for the nutrition of cardiomyocytes, wherein the reservoir is connected preferably fluidically to the at least one microfluidic channel and the at least one culture chamber and especially preferably the at least one pump is configured to deliver the nutrient solution to the at least one culture chamber, in particular via the at least one microfluidic channel.

The microfluidic device may be configured to increase the outflow of nutrient solution from the reservoir when there is strong activity of the cardiomyocytes, preferably by increasing the pumping frequency and/or pump lift and/or by pressurization of the reservoir. Furthermore, the microfluidic device may be configured to decrease the outflow of nutrient solution from the reservoir when there is weak activity of the cardiomyocytes, preferably by decreasing the pumping frequency and/or pump lift and/or by lowering the pressure on the reservoir.

The microfluidic channel may comprise at least one valve and/or at least one throttle. The at least one valve and/or the at least one throttle preferably comprise an elastic membrane, which is preferably arranged in at least one wall of the at least one microfluidic channel and especially preferably comprises or consists of plastic, quite especially preferably a plastic selected from the group consisting of thermosetting plastic, thermoplastic, elastomer and combinations thereof, especially a plastic selected from the group consisting of PC, PET, COC, PDMS, TPE and combinations thereof. Moreover, the elastic membrane may be suitable for being controlled by a pneumatic, thermopneumatic, electromagnetic, electrostatic, magnetic, chemical and/or piezoelectric force, wherein the valve and/or the throttle is preferably connected to at least one actuating channel (e.g. a pneumatic channel and/or a hydraulic channel) and/or at least one voltage source.

The at least one control device may be configured to control the at least one valve and/or the at least one throttle, based on activity of the cardiomyocytes detected by the at least one detector. In this case the at least one control device is preferably configured to at least partially open the at least one valve and/or the at least one throttle when there is strong activity of the cardiomyocytes. Furthermore, the at least one control device may be configured to at least partially close the at least one valve and/or the at least one throttle when there is weak activity of the cardiomyocytes.

The microfluidic device may comprise at least one oxygenator for oxygenation or deoxygenation of the liquid in the microfluidic device, preferably a gas-permeable membrane and/or a hollow fiber. Moreover, the microfluidic device may comprise an O₂ sensor for measuring the O₂ content of the liquid in the microfluidic device, preferably an O₂ sensor selected from the group consisting of optical O₂ sensor, electrochemical O₂ sensor and combinations thereof.

The at least one control device may further be configured to control the oxygenator as a function of the O₂ content measured via the O₂ sensor and/or as a function of the activity of the cardiomyocytes detected by the at least one detector.

In a preferred embodiment, the detector comprises or consists of an optical detector, preferably an optical detector configured to measure the calcium concentration. In the case of an optical detector, the culture chamber of the microfluidic device is transparent, at least in some zones, to light of the wavelength of the optical detector (preferably VIS spectrum). For example, the optical detector may comprise a microscope camera, which in particular is coupled to a real-time capable evaluating unit, wherein the evaluating unit is preferably configured to determine pharmacologically relevant parameters.

Moreover, the detector may comprise or consist of an electrical detector, preferably a multielectrode array (MEA). The electrical sensors are able to perform a run time measurement. An advantage with nonoptical detection is a lower hardware cost for image evaluation. In addition, the detector may comprise or consist of a mechanical detector, preferably a detector selected from the group consisting of strain gauges, tracking force microscope, spring beam and combinations thereof. In some respects the microfluidic device may be regarded in the case of these detectors as a cell-based (chemo-)(electro-)(opto-)mechanical transducer, which reacts to changes of the microenvironment, metabolic products or application of substances.

The detector may be configured to send signals about activity of the cardiomyocytes contained in the at least one first culture chamber to a data acquisition unit, wherein the data acquisition unit is preferably configured to record and evaluate the signals as a function of time.

In a preferred embodiment the microfluidic device comprises at least one device for influencing activity of the cardiomyocytes contained in the at least one first culture chamber. In this way it is possible for diseases of the cardiovascular system, e.g. cardiac insufficiency, heart rhythm disturbances, etc., to be produced artificially.

The device for influencing the activity of the cardiomyocytes preferably comprises or consists of an electrode, especially preferably at least one electrode selected from the group consisting of immersion electrode, planar electrode or a combination thereof. In particular, the device comprises or consists of a multielectrode array. Electrical contacting of the cardiomyocytes via this device offers the possibility of stimulating the heart tissue electrically, e.g. to simulate certain states such as physical strain. The advantage of multielectrode arrays (MEAS) is that they can also serve as electrical sensors, and thus can perform both stimulation of the cardiomyocytes and detection of their activity.

Furthermore, the device for influencing activity of the cardiomyocytes contained in the at least one first culture chamber may comprise or consist of at least one actuator, especially preferably an actuator selected from the group consisting of piezoelectric actuators, electromechanical actuators, pneumatic actuators, hydraulic actuators, surface tension actuators and combinations thereof.

In addition, the device for influencing activity of the cardiomyocytes contained in the culture chamber may comprise or consist of a device for supply or removal of gas, preferably a gas exchange membrane, wherein the device for supply or removal of gas preferably has a source of a gas selected from the group consisting of (pure) oxygen, air (e.g. compressed air), nitrogen, carbon dioxide and combinations thereof, in particular a source of a gas selected from the group consisting of air (e.g. compressed air), nitrogen, carbon dioxide and combinations thereof.

In an especially preferred embodiment, the microfluidic device comprises at least one access for supplying an (e.g. chemical and/or biological and/or biochemical) substance. This embodiment is advantageous for testing active pharmaceutical ingredients. Optionally this access opens directly into the at least one microfluidic channel, directly into the at least one first culture chamber and/or directly into the at least one reservoir. Supply of one or more chemical, biochemical or biological substances may therefore take place directly on the tissue or indirectly (e.g. via the inlets and outlets of the microfluidic device) via the flowing medium.

The microfluidic device may comprise at least one second microfluidic channel, which preferably branches off from the at least one first microfluidic channel upstream of the at least one first culture chamber and opens into the at least one first microfluidic channel downstream of the at least one first culture chamber.

Moreover, the microfluidic device may have at least one second culture chamber that contains biological cells which are different from cardiomyocytes. Preferably these cells are selected from the group consisting of liver cells, renal cells, nerve cells, fat tissue and combinations thereof, wherein the at least one second culture chamber especially preferably is connected fluidically to the at least one culture chamber, optionally via at least one second microfluidic channel. A throttle or a valve for influencing the flow may be comprised upstream of the second culture chamber. The microfluidic device may further have more than two culture chambers of this kind, wherein each of the culture chambers may have these properties. The culture chambers may also in each case be configured as separate subordinate microfluidic devices, which are connected to a microfluidic channel of the primary microfluidic device, e.g. via a plug-and-socket connection.

The microfluidic device may comprise at least one second detector, which is configured to detect an activity of the biological cells contained in the second culture chamber. The at least one second detector preferably comprises or consists of an optical detector and especially preferably is configured to send signals about activity of the biological cells contained in the at least one second culture chamber to a data acquisition unit, wherein the data acquisition unit is preferably configured to record and evaluate the signals as a function of time.

In a preferred embodiment the microfluidic device, preferably the at least one microfluidic channel, the at least one first culture chamber and/or at least one reservoir, contains blood or blood constituents, preferably blood cells, especially preferably cancer cells and/or immunocytes, especially metastasizing cancer cells and/or monocytes.

The at least one control device may comprise a flow velocity measuring device, preferably a particle image velocimetry apparatus, wherein the microfluidic device especially preferably is configured to control, on the basis of a flow velocity measured by the flow velocity measuring device, the at least one pump, preferably all pumps, of the microfluidic device. Moreover, the microfluidic device may especially preferably be configured to control, on the basis of a flow velocity measured by the flow velocity measuring device, at least one valve and/or at least one throttle, preferably all valves and/or all throttles, of the microfluidic device.

The microfluidic device may comprise or consist of plastic. The plastic preferably comprises or consists of at least one structured plastic film, which is laminated with at least one further, optionally unstructured, plastic film. Moreover, the plastic may be selected from the group consisting of thermosetting plastics, thermoplastics, elastomers and combinations thereof, especially preferably from a plastic selected from the group consisting of PC, PET, COC, PDMS, TPE and combinations thereof. In addition, the plastic may be structured at least partially by a method that is selected from the group consisting of laser structuring, cutting-plotting, hot embossing, milling, thermoforming, injection molding, soft lithography, 3D printing and combinations thereof.

The control device may be configured to adapt a distribution of liquid, solid and gaseous substances in the microfluidic device to a concrete requirement of cells or cell groups in the microfluidic device, preferably by selective control of the pump, of at least one valve, of at least one throttle and of an oxygenator, wherein selective control especially preferably takes place via a mathematical model, which in particular is stored in the microfluidic device, preferably in the control device. The mathematical model comprises a physical description of the flow and mass transfer in the subordinate or the primary microfluidic device or in the complete microfluidic device.

The microfluidic device may further comprise a device for detecting the flow velocity of fluids. The actual flow velocity of the fluid can thus be monitored. The flow velocity may be detected noninvasively from flowing particles or cells (e.g. blood constituents) by means of PIV with an adapted motion tracking module. However, invasive measurement (e.g. with a thermal anemometer) is also possible.

The microfluidic device may further comprise a device for detecting the O₂ content in fluids and a gas source for controlling the O₂ content in fluids. Enrichment of liquid in the microfluidic device with oxygen may thus take place if required.

The microfluidic device may have at least one further microfluidic device, with parallel or serial fluidic connection to the (first) microfluidic device (e.g. via just one individual fluidic connection, i.e. a single microfluidic channel). The fluidic connection preferably has at least one fluidic inlet and at least one fluidic outlet (e.g. in each case in the form of microfluidic channels). Therefore a fluid circuit may be formed between the (first) microfluidic device and the further microfluidic device. In this case the (first) microfluidic device may be a primary microfluidic device and the at least one further fluidic device may be a subordinate microfluidic device (and vice versa). Furthermore, the microfluidic device according to the invention may itself be divided for example into a subordinate part and a primary part. The at least one first culture chamber containing cardiomyocytes may then be comprised in a subordinate part of the microfluidic device and for example the at least one pump and/or the at least one detector in a primary part of the microfluidic device. If the at least one detector is arranged in the primary part, it may be in fluidic communication with the subordinate part. In the case of an optical detector, however, this fluidic communication is not necessary. A primary part of the microfluidic device or a primary, further microfluidic device may be configured in the form of a layer, which contacts a subordinate part of the microfluidic device or a subordinate, further microfluidic device (and vice versa).

Moreover, the microfluidic device may have at least two further microfluidic devices, with parallel or serial fluidic communication with the (first) microfluidic device and/or with one another (e.g. in each case via just one individual fluidic connection, i.e. a single microfluidic channel). The fluidic connection preferably has at least one fluidic inlet and at least one fluidic outlet (e.g. in each case in the form of microfluidic channels). A fluid circuit may therefore be formed between the (first) microfluidic device and the at least two further microfluidic devices. In this case the (first) microfluidic device may be a primary microfluidic device and the at least two further fluidic devices may in each case be subordinate microfluidic devices (and vice versa). Furthermore, at least one, optionally both, of the at least two further microfluidic devices according to the invention may themselves be divided for example into a subordinate part and a primary part. In this case at least one further culture chamber may contain cells that are not cardiomyocytes (e.g. cells selected from the group consisting of liver cells, renal cells, nerve cells, fat tissue and combinations thereof) and these may be contained in a subordinate part of the further microfluidic device and for example at least one part different from the further culture chamber in a primary part of the further microfluidic device. A primary part of the microfluidic device or primary, further microfluidic devices may in each case be configured in the form of a layer, which contacts a subordinate part of the microfluidic device or subordinate, further microfluidic devices (and vice versa).

It is further proposed to use the microfluidic device according to the invention for verifying whether a particular substance or metabolites thereof have an influence on the activity of cardiomyocytes, preferably for verifying whether the particular substance triggers tachycardia or bradycardia of the cardiomyocytes or gives rise to arrhythmia of the cardiomyocytes.

In addition it is proposed to use the microfluidic device according to the invention for verifying whether the mechanical, electrical and/or gas-induced influence (e.g. via an oxygenator) of activity of the cardiomyocytes has an influence on the activity of biological cells that are different from cardiomyocytes. Preferably it is verified whether and to what extent this influence has a negative or positive effect on the activity (viability) of the biological cells.

The subject matter of the invention will be explained in more detail on the basis of the following figures, without wishing to limit this to the specific embodiments shown here.

### LIST OF REFERENCES SYMBOLS

1.1: cardiomyocytes;

1.2: cells of another organ (e.g. liver cells);

2: microfluidic channel;

3: pump (e.g. micropump);

4.X: detector (e.g. multielectrode array);

4.1: optical detector (e.g. microscope);

4.2: electrical detector (e.g. multielectrode array (“MEA”));

4.3: mechanical detector (e.g. strain gauge);

5: control device;

6.X: data acquisition unit (e.g. for detecting cardiomyocyte activity);

6.1: bradycardia;

6.2: tachycardia;

7.1: pump parameters for influencing the flow (as signal of the feedback loop);

7.2: flow parameters for influencing the flow (as signal of the feedback loop);

7.3 gas composition for the oxygenator (as signal of the feedback loop);

8: supply of substance(s);

9: reservoir;

10: valve;

11: throttle;

12: elastic membrane;

13: electrical device for influencing the activity of the cardiomyocytes;

14: mechanical device for influencing the activity of the cardiomyocytes;

15: primary microfluidic device;

16.1: first subordinate microfluidic device;

16.2: second subordinate microfluidic device;

17: particles (e.g. blood constituents);

18.X: structured plastic film;

19: device for detecting the flow velocity of fluids;

20: device for detecting the O₂ content (e.g. O₂ sensor);

21: oxygenator;

22: fluidic interface;

23: actuating channel (e.g. channel to a gas source).

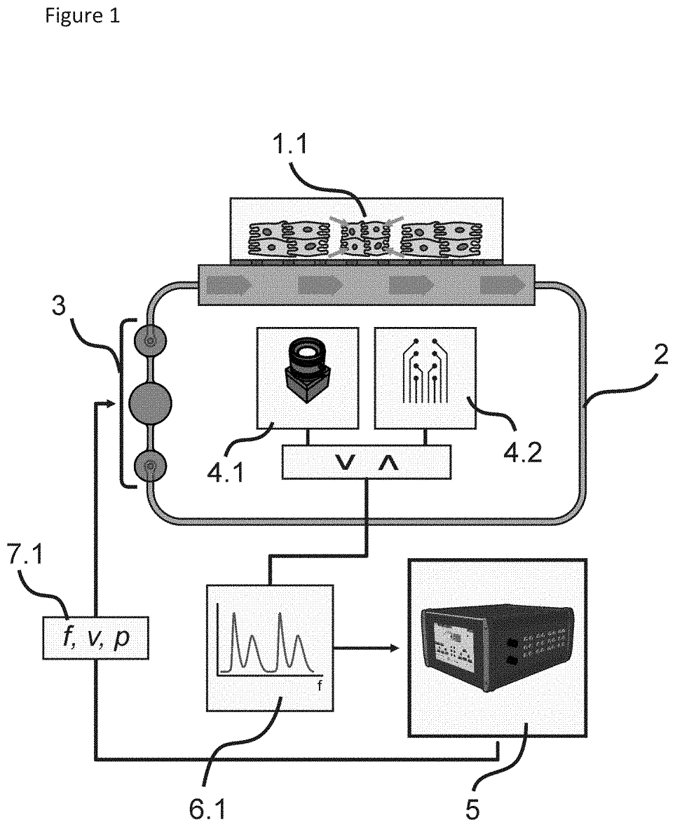

FIG. 1 shows a configuration of a microfluidic device for cell culture experiments as claimed in claim 1. In this embodiment, a microfluidic device is shown, which comprises a cell culture chamber with cardiomyocytes 1.1, a microfluidic channel 2 and a pump 3 as fluidic actuator. For readout of the cardiomyocyte activity, in this configuration an optical detector 4.1 and/or a multielectrode array 4.2 are arranged underneath the cell culture chamber. The signal picked up by the detectors 4.x is acquired by a data acquisition unit 6.x and relayed to a control device 5. According to the invention, in the control system 5 the signals received are processed and the pump parameters 7.1 are adapted for influencing the flow.

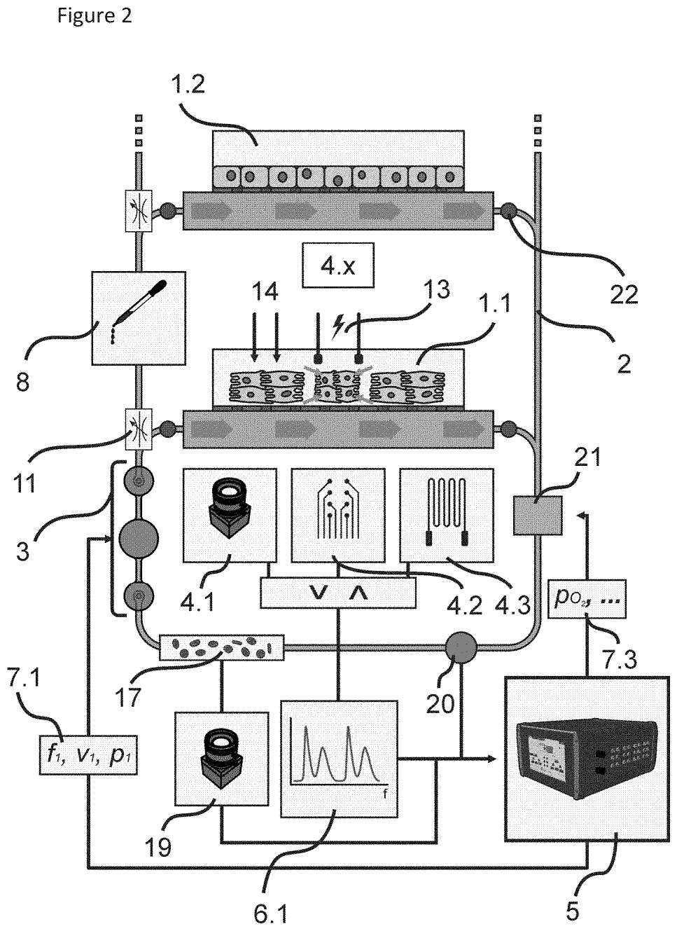

FIG. 2 shows a configuration of a microfluidic device for cell culture experiments as claimed in claim 1 and claim 22. In this embodiment, a microfluidic device is shown, which comprises at least one microfluidic channel 2 and a pump 3 as fluidic actuator. Furthermore, several subordinate microfluidic devices each with a cell culture chamber 1.x containing various cell types (cardiomyocytes 1.1, renal cells 1.2, etc.), are present. The at least two subordinate microfluidic devices are connected to one another and to the primary microfluidic device via defined fluidic interfaces 22. Furthermore, fluidic throttles 11 for volume flow control may be arranged before each channel branching. For readout of the cardiomyocyte activity or of physiological parameters of other cell types, in this configuration an optical detector 4.1 and/or a multielectrode array 4.2 and/or a strain gauge 4.3 may in each case be arranged underneath the cell culture chamber. Furthermore, an O₂ sensor 20 and an oxygenator 21 may be arranged within the microfluidic devices. For measuring the flow velocity, an optical detector 19 for detecting the movement of circulating particles or cells 17 may be arranged in the microfluidic device. The signal picked up by the detectors 4.x, 19 and 20 is acquired by a data acquisition unit 6.x and relayed to a control device 5. According to the invention, in the control system 5, the signals received are processed and the pump parameters 7.1 are adapted for influencing the flow and the gas composition for the oxygenator 7.3.

In this configuration, the cell culture chamber 1.1 further comprises an electrode 13 and/or an actuator 14 for influencing the activity of the cardiomyocytes.

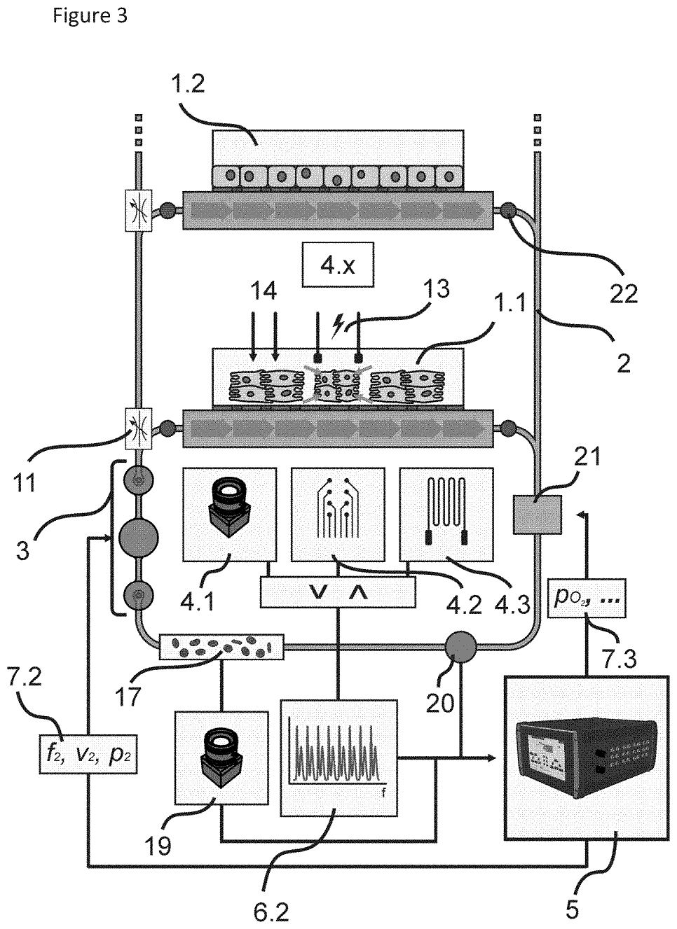

The microfluidic device presented may be used according to the invention for verifying whether a particular substance 8 or metabolites thereof have an influence on the activity of cardiomyocytes, preferably for verifying whether the particular substance triggers bradycardia 6.1 or tachycardia (FIG. 3) 6.2 of the cardiomyocytes or gives rise to arrhythmia of the cardiomyocytes.

FIG. 3 shows the microfluidic device from FIG. 2 after adding the substance 8. The flow parameter 7.2 and gas composition 7.3 are adapted appropriately on the basis of the altered activity 6.2 of the cardiomyocytes on adding the substance.

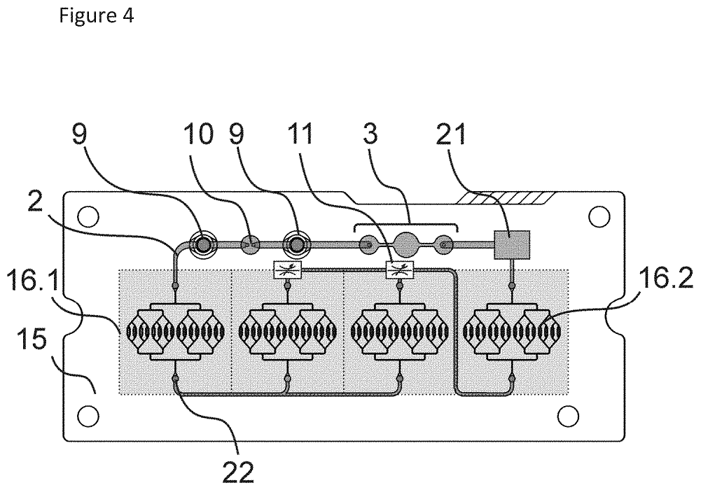

FIG. 4 shows a microfluidic device as claimed in claims 1 and 22. In this embodiment, consisting of a primary microfluidic device 15 comprising a pump 3, at least one microfluidic channel 2, two reservoirs 9, a valve 10, fluidic throttles 11 and an oxygenator 21, the primary microfluidic device 15 is connected via defined fluidic interfaces 22 with at least one first subordinate microfluidic device 16.1, in this case with at least one second subordinate microfluidic device 16.2.

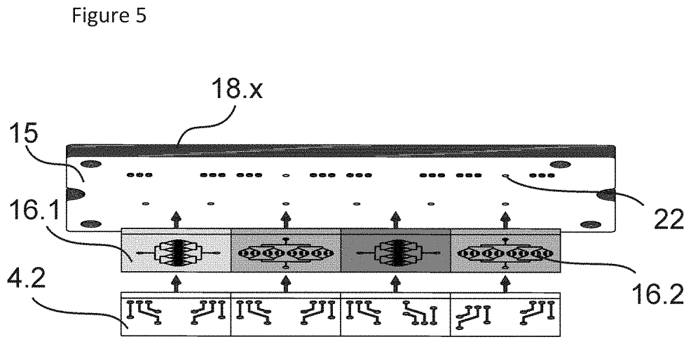

FIG. 5 shows the structure of the microfluidic device according to FIG. 4, comprising a primary microfluidic device 15, which consists of several laminated plastic films 18.x, and several subordinate microfluidic devices 16.1, 16.2. In this embodiment, the microfluidic device comprises several multielectrode arrays 4.x.

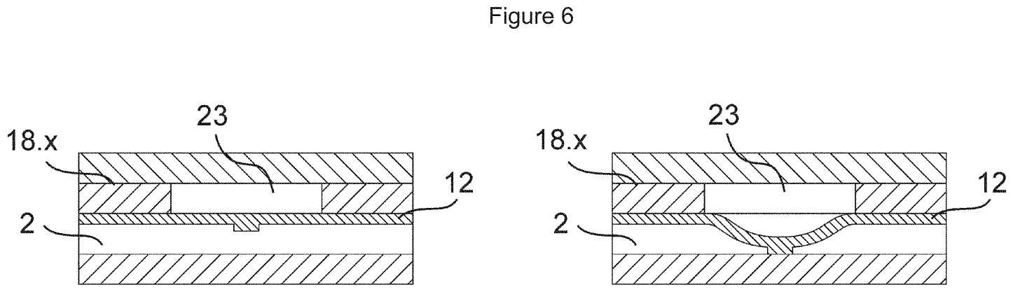

FIG. 6 shows an embodiment of the fluidic throttle, consisting of at least three (here four) laminated plastic films 18.x, a microfluidic channel 2 and an elastic membrane 12. In this embodiment, the elastic membrane may be deflected via an actuating channel 23. FIG. 6 shows the activated (deflected) and the nonactivated (nondeflected) state of the fluidic throttle.

1-23. (canceled)

24. A microfluidic device comprising
a) at least one first culture chamber containing cardiomyocytes;
b) at least one microfluidic channel;
c) at least one pump for pumping a liquid through the at least one microfluidic channel and the at least one culture chamber; and
d) at least one detector, which is configured to detect an activity of the cardiomyocytes contained in the culture chamber;
wherein the microfluidic device further comprises at least one control device which is configured to control the at least one pump based on the activity of the cardiomyocytes detected by the at least one detector.

25. The microfluidic device as claimed in claim 24, wherein the at least one control device is configured to:
i) increase the pumping capacity of the at least one pump when there is strong activity of the cardiomyocytes; and/or
ii) decrease the pumping capacity of the at least one pump when there is weak activity of the cardiomyocytes.

26. The microfluidic device as claimed in claim 25, wherein the at least one control device is further configured to
i) reduce or increase perfusion of the at least one first culture chamber containing cardiomyocytes; and/or
ii) reduce or increase perfusion of at least one second culture chamber in the microfluidic device.

27. The microfluidic device as claimed in claim 24, wherein the at least one first culture chamber contains cardiomyocytes, which form at least one heart muscle fiber.

28. The microfluidic device as claimed in claim 24, wherein the microfluidic device has at least one reservoir which contains a nutrient solution for the nutrition of cardiomyocytes.

29. The microfluidic device as claimed in claim 28, wherein the microfluidic device is configured to:
i) increase the outflow of nutrient solution from the reservoir when there is strong activity of the cardiomyocytes or
ii) reduce the outflow of nutrient solution from the reservoir when there is weak activity of the cardiomyocytes.

30. The microfluidic device as claimed in claim 24, wherein the microfluidic channel comprises at least one valve and/or at least one throttle.

31. The microfluidic device as claimed in claim 30, wherein the at least one control device is further configured to control the at least one valve and/or the at least one throttle based on activity of the cardiomyocytes detected by the at least one detector.

32. The microfluidic device as claimed in claim 24, wherein the microfluidic device comprises at least:
i) an oxygenator for oxygenation or deoxygenation of the liquid in the microfluidic device; and/or
ii) an O₂ sensor for measuring the O₂ content of the liquid in the microfluidic device.

33. The microfluidic device as claimed in claim 32, wherein the at least one control device is further configured to control the oxygenator as a function of the O₂ content measured by the O₂ sensor and/or as a function of the activity of the cardiomyocytes detected by the at least one detector.

34. The microfluidic device as claimed in claim 24, wherein the detector comprises:
i) an optical detector;
ii) an electrical detector; and/or
iii) a mechanical detector.

35. The microfluidic device as claimed in claim 24, wherein the detector is configured to send signals about activity of the cardiomyocytes contained in the at least one first culture chamber to a data acquisition unit.

36. The microfluidic device as claimed in claim 24, wherein the microfluidic device comprises at least one device for influencing activity of the cardiomyocytes contained in the culture chamber.

37. The microfluidic device as claimed in claim 24, wherein the microfluidic device comprises at least one access for supplying a chemical, biochemical or biological substance, wherein the access optionally opens directly into the microfluidic channel, directly into the at least one first culture chamber, and/or directly into the at least one reservoir.

38. The microfluidic device as claimed in claim 24, wherein the microfluidic device comprises at least one second microfluidic channel.

39. The microfluidic device as claimed in claim 24, wherein the microfluidic device has at least one second culture chamber that contains biological cells, which are different from cardiomyocytes.

40. The microfluidic device as claimed in claim 24, wherein the microfluidic device comprises at least one second detector, which is configured to detect an activity of the biological cells contained in the second culture chamber.

41. The microfluidic device as claimed in claim 24, wherein the microfluidic device contains blood or blood constituents.

42. The microfluidic device as claimed in claim 24, the at least one control device comprises a flow velocity measuring device.

43. The microfluidic device as claimed in claim 24, wherein the microfluidic device comprises a plastic.

44. The microfluidic device as claimed in claim 24, wherein the control device is configured to adapt the distribution of liquid, solid and gaseous substances in the microfluidic device to a concrete requirement of cells or cell groups in the microfluidic device.

45. The microfluidic device as claimed in claim 24, wherein the microfluidic device has at least one further microfluidic device, with parallel or serial fluidic communication with the microfluidic device.

46. A method comprising utilizing the microfluidic device of claim 24 in verifying whether
i) a particular substance or metabolites thereof have an influence on the activity of cardiomyocytes and/or
ii) the mechanical, electrical and/or gas-induced influence of activity of the cardiomyocytes has an effect on the activity of biological cells, which are different from cardiomyocytes.
